SEQUENCE LISTING (2 sequences)

SEQ ID NO: 1 - protein, 239 aa, Homo sapien
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 2 - protein, 236 aa, Rattus norvegicus
MAQAGRTGYDNREIVMKYIHYKLSQRGYEWDTGDEDSAPLRAAPTPGIFSFQPESNRTPAVHRDTAARTSPLRPLVANAGPALSPVPPVVHLTLRRAGDDFSRRYRRDFAEMSSQLHLTPFTARGRFATVVEELFRDGVNWGRIVAFFEFGGVMCVGSVNREMSPLVDNIALWMTEYLNRHLHTWIQDNGGWDAFVELYGPSMRPLFDFSWLSLKTLLSLALVGACITLGAYLGHK

(19) United States
(12) Patent Application Publication
(10) Pub. No.: US 2010/0189802 A1
(43) Pub. Date: Jul. 29, 2010

(54) Method for treatment of vascular hyperpermeability

(75) Inventors: Ed W. Childs, Temple, TX (US); W. Roy Smythe, Belton, TX (US)

Correspondence Address: CONLEY ROSE, P.C., 5601 GRANITE PARKWAY, SUITE 750, PLANO, TX, 75024, US

(73) Assignee: Scott and White Memorial Hospital and Scott, Sherwood, and Brindley Foundation, Temple, TX (US)

(21) Appl. No.: 12/693,788

(22) Filed: Jan. 26, 2010

Related U.S. Application Data
(63) Continuation-in-part of application No. 12/229,127, filed on Aug. 20, 2008.
(60) Provisional application No. 60/965,586, filed on Aug. 21, 2007.

(51) Int. Cl. A61K 38/17; A61K 35/16; A61K 38/13; A61K 38/06; A61K 38/18

(52) U.S. Cl. 424/530; 514/11; 514/12; 514/18

(57) ABSTRACT

A method comprising preparing a vascular hyperpermeability treatment composition comprising an apoptosis regulating protein, a antioxidant, a mitochondrial modulator, a biological effector molecule, or combinations thereof in a form deliverable to a mammal; whereby an effective amount of the composition raises the threshold for apoptosis and/or prevents or lessens the occurrence of vascular hyperpermeability. A method comprising preventing hyperpermeability associated with hemorrhagic shock via preparing a composition in a form deliverable to a mammal, the composition comprising an apoptosis regulating protein, a antioxidant, a mitochondrial modulator, a biological effector molecule, or combinations thereof; and raising the threshold for apoptosis in a mammal's endothelial cells by administering an effective amount of said antioxidant; whereby said raising the threshold for apoptosis prevents said hyperpermeability.

### CROSS-REFERENCE TO RELATED APPLICATIONS

This application is a continuation-in-part of U.S. patent application Ser. No. 12/229,127 filed on Aug. 20, 2008 and entitled “Method for Treatment of Vascular Hyperpermeability,” which claimed priority to U.S. Provisional Patent Application No. 60/965,586, filed on Aug. 21, 2007 and entitled “Method for Treatment of Vascular Hyperpermeability,” each of which is hereby incorporated by reference herein in its entirety.

### STATEMENT REGARDING FEDERALLY SPONSORED RESEARCH OR DEVELOPMENT

This invention was made in part with government support under Grant No. 5K01HL76815-3 awarded by the National Institutes of Health and Grant No. HL-03-011 awarded by the National Institutes of Health. The government has certain rights in the invention.

### REFERENCE TO A SEQUENCE LISTING

In compliance with 37 C.F.R. §1.52(e)(5), an electronic CRF of the sequence listing is filed herewith: file name: BCLsequencelisting_ST25.txt; size 5 KB; created on: Jan. 20, 2010; using PatentIn-3.5, is hereby incorporated by reference in its entirety. The data in the Computer Readable Form of the Sequence Listing submitted herewith contains no new matter.

### FIELD OF THE INVENTION

The invention disclosed herein is a method for treatment of vascular hyperpermeability.

### BACKGROUND OF THE INVENTION

Trauma is a leading cause of death for individuals under the age of 44. Individuals who have suffered extensive trauma exhibit hemorrhagic shock which is usually treated with fluids and medicines to maintain blood pressure. Despite the best efforts, patients die because of the inability to maintain sufficient blood pressure to properly perfuse the major organs of the body. One of the causes of death is vascular hyperpermeability secondary to hemorrhagic shock. Vascular hyperpermeability is the process by which the fluid portion of blood leaks out of the vascular structures into the tissues of the body. This leakage of fluid may cause the tissues to swell or the development of edema. Fluid congestion of the tissues and organs may develop which may thereby result in organ failure. Vascular hyperpermeability may also cause some of the fluid administered intravenously during resuscitation efforts to leak out of the vascular system and into the surrounding tissues. This leakage of the intravenous fluids from the vascular system contributes to the edema and organ failure. Leakage of intravenous fluids may also make it difficult to maintain an effective blood pressure and perfusion of the organs with oxygenated blood.

Apoptosis or programmed cell death is a normal process in which old cells die and are replaced with new cells. Apoptosis is an orderly process of cell death as distinguished from necrosis which is the result of acute cellular injury. In the body, cells are constantly dying and being replaced. Cells die when they are damaged beyond repair, infected with a virus or undergo stress, for example, starvation. These cells die, are removed, and are replaced with new cells. In some circumstances, the balance between old cell death and new cell division is out of balance. When cell division occurs at a rate faster than cell death, tumors may develop. When cell division occurs at a rate slower than cell death, a disorder or disruption in the structure and function of the affected tissue may occur.

There are several proteins involved in regulation of apoptosis. The process of apoptosis is managed by extracellular and intracellular signals. Nonlimiting examples of extracellular signals include hormones, growth factors, and cytokines, which may cross the cell membrane and thereby affect a response. The intracellular signal may be initiated by a cell under stress and may result in cell death. Before cell death can occur one or more of the signals mentioned above must be connected to the apoptotic pathway by way of regulatory proteins.

One set of proteins targets the mitochondria, as will be discussed below. The mitochondrion is a cell organelle which is essential to the life of the cell. The main function of the mitochondrion is to enable aerobic respiration, or energy production, by the cell. Disruption of the mitochondrion quickly results in cell death. The apoptotic regulatory proteins affect the permeability of the mitochondrion and cause swelling of the cell through the development of pores in the membrane. Cytochrome c is released from the mitochondrion due to the increased permeability of the outer mitochondrial membrane and serves a regulatory function as it precedes morphological changes in the cell associated with apoptosis. Once cytochrome c is released, it binds with another regulatory protein and adenosine triphosphate (ATP), which then binds to pro-caspase-9 to create an apoptosome. The apoptosome cleaves the pro-caspase to its active form of caspase-9, which in turn activates the effector, caspase-3. Caspase-3 is an enzyme which cleaves other proteins to actually start the process of intrinsic apoptosis.

Mitochondrial permeability is subject to regulation by various proteins of the Bcl-2 family of proteins. The Bcl-2 proteins are able to promote or inhibit apoptosis by either direct action on mitochondrial permeability or indirectly through other proteins. Some of the Bcl-2 proteins can stop apoptosis even if cytochrome c has been released by the mitochondrion. The Bcl-2 proteins are frequently referred to as intrinsic mitochondrial regulatory proteins.

Hemorrhagic shock and resuscitation activates a cascade of inflammatory mediators, resulting in tissue damage, multiple organ dysfunction, and if unabated, death. Ischemia associated with shock, and the resulting oxidative stress during resuscitation, contribute to the development of this systemic inflammatory response. The oxidative stress caused by ischemia/reperfusion results in an increase in reactive oxygen species (ROS) generation which activates leukocytes and damages endothelial cells. Activation of ROS that subsequently damages the endothelium has been shown to increase microvascular permeability. It has been demonstrated that ROS are generated following hemorrhagic shock. (Childs E, Tharakan B, Hunter F, Isong M, Liggins N. Mitochondrial complex III is involved in proapoptotic BAK-induced microvascular endothelial cell hyperpermeability. Shock 2008. 29(5) 636-641; Tharakan B, Hunter F, Smythe W R, Child E. Alpha-lipoic acid attenuates hemorrhagic shock-induced apoptotic signaling and vascular hyperpermeability. Shock 2008. 30(5) 571-577; Tharakan B, Whaley J, Hunter F, Smythe W R, Childs E. Deprenyl inhibits vascular hyperpermeability following hemorrhagic shock. Shock 2009). In addition, it has been shown that the endothelium is an important source of ROS generation. Since ROS are by-products of oxidative phosphorylation, most intracellular ROS are produced by the mitochondria. ROS produced at sites other than mitochondria have been reported to be involved in some apoptotic systems, but it is widely accepted that mitochondria are the predominant source of ROS produced in the “intrinsic” mitochondrial apoptotic cascade.

Apoptosis can also be regulated by certain cell-specific growth factors. For example, the endothelial cell growth factor, angiopoietin-1, has been observed to stop apoptosis and prevent vascular hyperpermeability and edema following hemorrhagic shock. The angiopoietin-1 protein prevents apoptosis of endothelial cells by regulating the apoptotic signaling pathway leading to endothelial cell death and vascular hyperpermeability (Childs et. al. Am J. Physiol Heart Circ Physiol 2008. 294:H2285-2295). Treatment of traumatized animals with angiopoietin-1 shows that this compound is a potent inhibitor of vascular hyperpermeability and apoptosis.

If apoptosis continues to cell death, several morphological features are evident:

1. Cell shrinkage and rounding due to the breakdown of the proteinaceous cytoskeleton by enzymes.
2. The cytoplasm of the cell appears dense, and the organelles appear tightly packed.
3. Chromatin undergoes condensation into compact patches against the nuclear envelope.
4. The nuclear envelope becomes discontinuous and the DNA inside is fragmented.
5. The cell membrane shows irregular buds or blebs.
6. The cell breaks apart into several apoptotic bodies which are removed by phagocytosis.

By this process the dead and dying cells and their contents are removed in an orderly fashion and replaced with new, viable cells. There are currently no available methods or treatments to inhibit apoptosis of endothelial cells following trauma and shock. The ability to inhibit apoptosis of endothelial cells following shock would diminish conditions such as edema caused by vascular hyperpermeability resulting from the death of the endothelial cells. What is needed in the art is a method to protect the endothelial cells from apoptotic death and prevent edema from developing after the patient has suffered trauma sufficient to induce hemorrhagic shock and vascular hyperpermeability.

### SUMMARY OF INVENTION

Disclosed herein is a method comprising preparing a vascular hyperpermeability treatment composition comprising an apoptosis regulating protein, an antioxidant, a mitochondrial modulator, a biological effector molecule, or combinations thereof, in a form deliverable to a mammal; whereby an effective amount of the composition raises the threshold for apoptosis and/or prevents or lessens the occurrence of vascular hyperpermeability.

Also disclosed herein is a method comprising preventing hyperpermeability associated with hemorrhagic shock via preparing a composition in a form deliverable to a mammal, the composition comprising an apoptosis regulating protein an antioxidant, a mitochondrial modulator, a biological effector molecule, or combinations thereof; and raising the threshold for apoptosis in a mammal's endothelial cells by administering an effective amount of said antioxidant; whereby said raising the threshold for apoptosis prevents said hyperpermeability.

Also disclosed herein is a pharmaceutical composition comprising α-lipoic acid, angiopoietin 1, Cyclosporin-A, an anti-apoptotic Bcl-2 protein, or combinations thereof.

Also disclosed herein is a pharmaceutical composition comprising: an antioxidant, a mitochondrial modulator, a intrinsic apoptosis regulating protein, an endothelial growth factor, or combinations thereof effective for diminishing hyperpermeability by modulating the apoptotic signaling associated with hemorrhagic shock in mammalian endothelial cells; and a transfection agent effective to achieve delivery of at least a portion of the composition to the endothelial cells.

Also disclosed herein is an article of manufacture comprising a pharmaceutical composition.

Also disclosed herein is a method comprising modulating mitochondrial apoptotic signals in endothelial cells associated with hemorrhagic shock via: preparing a composition in a form deliverable to a mammal, the composition comprising an antioxidant, a mitochondrial modulator, an intrinsic apoptosis regulating protein, an endothelial growth factor, or combinations thereof; and providing said composition to the mammal in an amount effective to raise the threshold for apoptosis associated with hemorrhagic shock in the endothelial cells of the mammal; whereby providing said composition modulates the activity of the mitochondrial apoptotic signals and diminishes hyperpermeability associated with apoptosis.

Also disclosed herein is a method for prolonging an effective therapeutic time period of intrinsic apoptosis regulating proteins to raise the threshold of apoptosis in mammals with hemorrhagic shock comprising steps of binding apoptosis regulating proteins to another compound; administering said apoptosis regulating protein bound to another compound to said mammal in hemorrhagic shock; whereby said apoptosis regulating protein bound to another compound attenuates apoptosis of endothelial cells for a longer period of time resulting in diminished vascular hyperpermeability.

Also disclosed herein is a method for delivery of apoptosis regulating proteins to specific receptors on cell membranes of endothelial cells of mammals with hemorrhagic shock comprising steps of preparing an antibody specific to said specific receptor on the cell membrane of endothelial cells; attaching said apoptosis regulating protein to said antibody; administering said apoptosis regulating protein attached to said antibody to said mammal with hemorrhagic shock:

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE SEVERAL VIEWS OF THE DRAWINGS

A better understanding of the invention disclosed herein may be had by review of the following drawings wherein:

FIG. 1 is a bar graph showing the attenuation of hemorrhagic shock-induced vascular hyperpermeability by Bcl-xl administered before, during and after the onset of shock;

FIG. 2 is a graph showing the attenuation of hyperpermeability induced by Bcl-xl given during resuscitation following 60 minutes of shock;

FIG. 3 is a graph showing the attenuation of hyperpermeability induced by Bcl-xl given during the shock period;

FIG. 4 is a graph showing the attenuation of hyperpermeability induced by Bcl-xl when given prior to the induction of shock;

FIG. 5 is a bar graph showing the diminution in release of cytochrome c following administration of Bcl-xl;

FIG. 6 is a bar graph showing the diminution in hemorrhagic shock-induced caspase-3 activity by Bcl-xl administration;

FIG. 7 is a graph showing the elimination of vascular permeability by the administration of Cyclosporin-A prior to the onset of hemorrhagic shock;

FIG. 8 is a bar graph showing the diminution in cytochrome c release following the onset of hemorrhagic shock by administration of Cyclosporin-A;

FIG. 9 is a bar graph showing the diminution in hemorrhagic shock-induced caspase-3 activity by administration of Cyclosporin-A.

### DETAILED DESCRIPTION

The invention disclosed herein describes one or more compositions for treatment of vascular hyperpermeability and prevention of edema in trauma patients by attenuation of the process of apoptosis of endothelial cells lining the structures of the vascular system. Further, the invention disclosed herein describes one or more methods for treatment of vascular hyperpermeability and prevention of edema in trauma patients by attenuation of the process of apoptosis of endothelial cells lining the structures of the vascular system. Attenuation of apoptosis as disclosed in the present invention may have the effect of decreasing vascular permeability by protecting the endothelial cell barrier from leakage of fluid into the interstitial space caused by apoptosis.

In an embodiment, compositions for the treatment of vascular hyperpermeability, hereinafter denoted vascular hyperpermeability treatment compositions (VHTC), may comprise an apoptosis-regulating protein, an antioxidant, a pharmaceutically active compound, a biological effector molecule, or combinations thereof. Such VHTCs may reduce, inhibit and/or prevent the various cellular events described herein following an adverse event (e.g., hemorrhagic shock) that lead to increased cellular apoptosis (e.g. apoptosis of endothelial cells). In an embodiment, the VHTC comprises one or more agents that function to attenuate the mitochondrial permeability.

One of ordinary skill in the art could readily envision any number of factors, events, and/or illnesses that may result in an organism experiencing vascular hyperpermeability. Nonlimiting examples of such factors, events, and/or illnesses have been disclosed previously herein. For example, vascular permeability by any measure is dramatically increased in acute and chronic inflammation, cancer, and wound healing. This hyperpermeability is mediated by acute or chronic exposure to vascular permeabilizing agents of the type described previously herein. The disclosure hereinafter will refer to vascular hyperpermeability as a result of hemorrhagic shock however other events resulting in vascular hyperpermeability are also contemplated.

In an embodiment, the VHTC comprises an apoptosis-regulating protein. In an embodiment, the apoptosis-regulating protein may be provided to attenuate the intrinsic pathway leading to apoptosis to thereby reduce vascular permeability and edema associated with hemorrhagic shock, as will be discussed in greater detail herein. In another embodiment, an apoptosis-regulating protein may be provided to attenuate the extrinsic pathway leading to apoptosis to thereby reduce vascular permeability and edema associated with hemorrhagic shock, as will be discussed in greater detail herein. Hereinafter, all proteins suitable for use in this disclosure are understood to be isolated and/or purified proteins. As used herein, the terms “isolated” or “purified” protein and/or polypeptide refer to a protein and/or polypeptide which may be substantially free of other cellular material or culture medium when produced by recombinant techniques or substantially free of chemical precursors or other chemicals when chemically synthesized. As used herein, “substantially free” refers to the amount in which other components that do not adversely affect the properties of the polypeptides, compositions, and/or organisms to which the compositions are introduced may be present. For example, the proteins and/or polypeptides of the disclosed herein may be greater than about 70% pure, alternatively greater than about 75%, 80%, 85%, 90%, 95%, or 99% pure.

As used herein, the term “protein” refers to an organic compound comprising at least twenty amino acids arranged in a linear or substantially linear chain and joined by peptide bonds between the carboxyl group and the amino group of adjacent amino acid residues without regard to whether the protein was naturally or artificially synthesized and also without regard to post-translational modification of the protein, secondary, tertiary, or quaternary structure. A peptide bond is the sole covalent linkage between amino acids in the linear backbone structure of all peptides, polypeptides or proteins. The peptide bond is a covalent bond, planar in structure and chemically constitutes a substituted amide. An “amide” is any of a group of organic compounds containing the grouping —CONH—. As used herein, the term “peptide” is a compound that includes two or more amino acids linked together by a peptide bond. As used herein, the term “polypeptide” is a compound that includes three or more amino acids linked together by a peptide bond.

The apoptosis-regulating protein and/or polypeptide may be isolated and/or purified using techniques known to one of ordinary skill in the art. For example, the polypeptide may be produced from a recombinant nucleic acid. As will be understood by those of ordinary skill in the art and as used herein, a recombinant nucleic acid is a nucleic acid produced through the addition of relevant DNA into an existing organism's genome. In an embodiment, the VHTC comprises an apoptosis-regulating protein which is obtained by chemical synthesis. As will be understood by those of ordinary skill in the art and as used herein, a protein may be synthesized by chemical means in a process involving the chemical ligation of peptides. Not seeking to be bound by any particular theory, a protein may be chemically synthesized via the chemical joining of amino acids. The VHTC may comprise a mixture of apoptosis regulating proteins that are obtained using standard isolation and/or purification techniques and apoptosis regulating proteins obtained via chemical synthesis.

In an embodiment, the apoptosis-regulating protein comprises an intrinsic apoptosis regulatory protein. An intrinsic apoptosis regulatory protein may comprise any protein suitable for impacting the mitochondrial outer membrane permeability and thereby regulating the onset of apoptosis of endothelial cells. Not to be bound by theory, the intrinsic apoptosis regulatory protein may function to (1) reduce the mitochondrial outer membrane permeability following an event that may lead to the onset of vascular hyperpermeability, decreasing the incidence of endothelial cell apoptosis; (2) inactivate the inner mitochondrial permeability transition pore (MPTP) and prevent the formation of the mitochondrial apoptosis induced channel (MAC) which would inhibit the release of cytochrome c into the cytosol, thus preventing or lessening the occurrence of apoptosis; or both.

In an embodiment, the apoptosis-regulating protein comprises an apoptosis inhibiting protein. As used herein, “apoptosis inhibiting protein” refers to a protein which may inhibit or otherwise impede an effector molecule (e.g., another protein, a cell signaling transducer, a hormone, the like, or combinations thereof) which functions to promote the apoptotic pathway.

In an embodiment, the intrinsic apoptosis-regulating protein is an anti-apoptotic member of the Bcl-2 family of proteins. As described above, the Bcl-2 family of proteins is highly conserved, regulatory proteins for modulating the permeability of the membrane of mitochondrion. These proteins are encoded by genes located on human chromosome 13 and received their name from the cell in which they were first discovered, B cell leukemia. In an embodiment, the Bcl-2 family of proteins comprises various antiapoptotic proteins.

As used herein, “anti-apoptotic” shall mean a molecule tending to prevent or decrease the occurrence of apoptosis. Nonlimiting examples of antiapoptotic Bcl-2 proteins include, the Bcl-xL protein, the MCL-1 protein, the A-1 protein, and the Bcl-w protein. Hereinafter, anti-apoptotic Bcl-2 family members are collectively referred to as aa-Bcl2. It is contemplated that other antiapoptotic members of the Bcl-2 family not yet identified but which function to downregulate the intrinsic apoptotic pathway may also be included in the VHTC. Further, it is to be understood that other non-Bcl2 proteins that function to reduce and/or inhibit the apoptotic pathway (e.g. through attenuation of the mitochondrial outer membrane permeability) may be utilized in the VHTC compositions of this disclosure. Such proteins may function to mitigate endothelial cell apoptosis and thus reduce and/or prevent the onset of vascular hyperpermeability. Such anti-apoptotic proteins may be chosen by one of ordinary skill in the art with the aid and benefit of this disclosure. The remainder of the disclosure will focus on the use aaBcl-2 proteins in the VHTC although other proteins of the type described herein are also contemplated.

In an embodiment, the aa-Bcl2 comprises a polypeptide having the amino acid sequence set forth in SEQ ID NO: 1. Alternatively, the aa-Bcl2 comprises a polypeptide having the amino acid sequence identified as SEQ ID NO: 2. Hereinafter the polypeptide having the amino acid sequence set forth in SEQ ID NO:1 is referred to as human-Bcl while the polypeptide having the amino acid sequence set forth in SEQ ID NO:2 is referred to as rat-Bd. In an embodiment the aa-Bcl2 comprises a functional derivative of human-Bd. In an embodiment the aa-Bcl2 comprises a functional derivative of rat-Bcl.

As used herein, a “functional derivative” is a compound that possesses a biological activity (either functional or structural) that is substantially similar to the biological activity of the protein of interest (e.g., human or rat aa-Bcl). The term “functional derivatives” is intended to include the “fragments,” “variants,” “degenerate variants,” “analogs” and “homologs” or “chemical derivatives” of protein of interest (e.g., aa-Bcl). The term “fragment” is any polypeptide subset of the protein of interest (e.g., aa-Bcl). The term “variant” is meant to refer to a molecule substantially similar in structure and function to either the entire protein of interest (e.g., aa-Bcl) molecule or to a fragment thereof. A molecule is “substantially similar” to the protein of interest (e.g., aa-Bcl) if both molecules have substantially similar structures or if both molecules possess similar biological activity. Therefore, if the two molecules possess substantially similar activity, they are considered to be variants even if the structure of one of the molecules is not found in the other or even if the two amino acid sequences are not identical. The term “analog” refers to a molecule substantially similar in function to either the entire protein of interest molecule (e.g., aa-Bcl) or to a fragment thereof. The term “chemical derivative” describes a molecule that contains additional chemical moieties which are not normally a part of the base molecule. Such moieties may improve the solubility, half-life, absorption, etc of the base molecule. Alternatively the moieties may attenuate undesirable side effects of the base molecule or decrease the toxicity of the base molecule. Examples of such moieties are described in a variety of texts, such as Remington's Pharmaceutical Sciences.

It is to be understood that the present disclosure contemplates the use of any functional derivative of any protein disclosed herein. Such functional derivatives are intended to include the “fragments,” “variants,” “degenerate variants,” “analogs” and “homologs” or “chemical derivatives” of any protein described as being suitable for use in the VHTC. The terms “fragments,” “variants,” “degenerate variants,” “analogs”, “homologs” and “chemical derivatives” are intended to retain their general definition as set forth previously herein with respect to the specific protein being disclosed.

In an embodiment, the VHTC comprises an extrinsic apoptosis regulating protein. Such proteins may function to downregulate the occurrence of apoptosis via mechanisms associated with the extrinsic apoptotic pathway. In some embodiments, the VHTC may comprise both extrinsic apoptosis regulating proteins and intrinsic apoptosis regulating proteins.

In an embodiment, the apoptosis-regulating protein is present in the VHTC in a pharmaceutically effective amount.

In an embodiment, the VHTC comprises an antioxidant of the type previously described herein. Any suitable antioxidant capable of reacting with and thereby lessening the reactivity of a ROS may be employed as the antioxidant of the VHTC. In an embodiment, the VHTC comprises a-lipoic acid, ascorbic acid, glutathione, uric acid, carotenoids (e.g., 3-Carotene and retinol), a-tocopherol, ubiquinol (e.g., Coenzyme Q10), deprenyl, or combinations thereof. In an embodiment, the antioxidant is present in the VHTC in a pharmaceutically effective amount.

In an embodiment, the VHTC comprises a mitochondrial modulator. The mitochondrial modulation may to modulate mitochondrial membrane permeability. In some embodiments, the mitochondrial modulator is an immunomodulatory agent. Nonlimiting examples of pharmaceutical compounds suitably employed in the invention disclosed herein include Cyclosporin-A, tacrolimus (also known as FK-506, Prograf®, Adragraf® or Protopic®), other mTOR proteins, such as isrolimus (rapamycin; Rapamune®), temsirolimus (Torisel®); or combinations thereof.

Not seeking to be bound by any particular theory, the mitochondrial modulator may function to attenuate (e.g., reduce) endothelial cell apoptosis, thereby inhibiting or preventing the onset of vascular hyperpermeability. Not seeking to be bound by any particular theory, the mitochondrial modulator may decrease the response of at least a portion of the immune system of a subject to which a VHTC is administered, thereby lessening the probability that the subject's immune system will reject the VHTC (e.g., the protein). In an embodiment, the mitochondrial modulator is present in the VHTC in a pharmaceutically effective amount.

In an embodiment, the VHTC comprises a biological effector molecule. Not seeking to be bound by any particular theory, the biological effector molecule may directly or indirectly stimulate angiogenesis, that is, the growth and development of blood vessels from preexisting blood vessels, or otherwise lessen vascular hyperpermeability by contributing to vasculature proliferation. In embodiments, the biological effector molecule may comprise a molecule which will elicit biological responses including but not limited to gene activation, cell proliferation, cell differentiation, and matrix dissolution thereby leading to mitogenic activity, that is, cell division and proliferation. Such biological responses may further include stimulation of regulatory cascades leading to angiogenesis, cellular migration, and/or degradation of matrix metalloproteinase (MMP), thus leading to capillary formation.

In various embodiments, the biological effector molecule comprises a protein, a glycoprotein, a cell-surface binding molecule, a cell transport molecule, a cell-signaling molecule, a receptor molecule, a gene product, or combinations thereof. The biological effector molecule may further comprise a precursor for a protein, glycoprotein, cell-surface binding molecule, cell transport molecule, cell-signaling molecule, receptor molecule, gene product, or combinations thereof. The biological effector molecule may further comprise a transcriptional enhancer for a protein, glycoprotein, cell-surface binding molecule, cell transport molecule, cell-signaling molecule, receptor molecule, gene product, or combinations thereof.

In an embodiment, the biological effector molecule comprises an endothelial growth factor. Alternatively, the biological effector molecule comprises angiopoietin-1. Not seeking to be bound by any particular theory, angiopoietin-1 may lessen vascular hyperpermeability by disrupting the signaling pathway by which apoptosis is initiated and sustained. By disrupting the apoptotic signaling pathway, the administration of angiopoietin-1 may lessen the occurrence of apoptosis of endothelial cells and thereby lessen vascular hyperpermeability. In an embodiment, the biological effector molecule is present in the VHTC in a pharmaceutically effective amount.

In an embodiment, the VHTC may further comprise one or more inhibitors of the apoptotic pathway. In another embodiment, the VHTC may further comprise one or more inhibitors of proapoptotic proteins such as for example BAK, BAX, and BOK.

In an embodiment, the VHTCs of this disclosure may be a component in a pharmaceutical composition wherein the composition is to be administered to an organism experiencing an undesired condition (e.g., vascular hyperpermeability) and act as a therapeutic agent for treatment of the undesired condition. Herein “treatment” refers to an intervention performed with the intention of preventing the development or altering the pathology of the undesirable condition. Accordingly “treating” refers both to therapeutic treatments and to prophylactic measures. In an embodiment, administration of therapeutic amounts of compositions of the type described herein to an organism confers a beneficial effect on the recipient in terms of amelioration of the undesirable condition. In an embodiment, the VHTCs may be used in conjunction with other therapeutic methods to effect the treatment of an undesirable condition. The VHTC may additionally comprise a pharmaceutically acceptable carrier or excipient. As used herein, “pharmaceutically acceptable carrier” is intended to include any and all solvents, dispersion media, coatings, antibacterial and anti-fungal agents, isotonic and absorption delaying agents, and the like, compatible with pharmaceutical administration. The use of such media and agents for pharmaceutically active substances is well known in the art.

In an embodiment, the VHTC's of this disclosure may be advantageously utilized in conjunction with conventional means and methods of treating a patient experiencing or at risk for vascular hyperpermeability. For example, a conventional method for the treatment of vascular hyperpermeability may comprise the administration of fluids (e.g., plasma) to a patient experiencing hemorrhagic shock. In such an embodiment, co-administration of the VHTC and blood plasma may decrease the amount of blood plasma which is necessarily administered to such a patient.

In an embodiment, a method for the treatment of vascular hyperpermeability may comprise administering a therapeutic amount of a VHTC of the type described herein. Herein “therapeutic amounts” refers to the amount of the composition necessary to elicit a beneficial effect. As will be recognized by one of skill in the art, administration of the VHTC may be by any suitable means. Non-limiting examples of such means of administering the composition include topical (e.g., epicutaneous), enteral (e.g., orally, via a gastric feeding tube, via a duodenal feeding tube, rectally), or parenteral, or combinations thereof. In a specific embodiment, administration of the composition may be by intravenous injection, intraarterial injection, intramuscular injection, intracardiac injection, subcutaneous injection, intraperitoneal injection, intraperitoneal infusion, transdermal diffusion, transmucosal diffusion, intracranial, intrathecal, or combinations thereof.

Although the combinations of agents comprising the VHTC are described herein as a single, unitary composition, it is contemplated that these components need not be administered in the form of a single unitary compound. That is, it is hereby contemplated that components of the VHTC may be administered individually or in concert. It is further contemplated that the different components of the VHTC need not be administered via a single route of administration. Thus, the following disclosure is meant to apply not only in the circumstance where the components of the VHTC are administered as a single, unitary composition, but also any situation in which components of the VHTC are utilized in concert to for the treatment of vascular hyperpermeability. For example, in an embodiment, a first component of the vascular hyperpermeability composition may be administered to the patient shortly after the patient experiences an undesirable condition. Thereafter, the patient may be administered additional components of the VHTC in subsequent time periods that may span hours, days, or weeks following the initial administration of a VHTC component.

In an embodiment, the components of the VHTC may be administered sequentially. In yet another embodiment, the components of the VHTC may be administered simultaneously. In an embodiment, the order in which the components of the VHTC are administered may be any order which will facilitate the goals or necessities of the user and depend upon a number of factors.

In an embodiment, a VHTC may suitably be administered therapeutically. As used herein therapeutic administration refers to the administration of a VHTC to a patient after or during a course of time in during which the patient experiences an undesirable condition. Nonlimiting examples of scenarios in which a VHTC may be administered to a patient therapeutically include prior to, coincident with and/or after surgery, after a medical treatment, or following a circumstance in which the patient may have experienced some form of trauma.

In an alternative embodiment, a VHTC may suitably be administered prophylactically. As used herein, prophylactic administration refers to the administration of a VHTC to a patient prior to the patient experiencing an undesired condition. Nonlimiting examples of scenarios in which a VHTC may be administered to a patient prophylactically include prior to, coincident with, and/or after surgery, prior to a medical treatment, or prior to a circumstance in which the patient to whom the VHTC is administered may experience some form of trauma.

In an embodiment, a VHTC may suitably be administered therapeutically and prophylactically. In an embodiment, the modes of treatment described herein may be utilized at least once, alternatively multiple times, throughout the course of a treatment regime. As will be understood by those of skill in the art, the number of times a patient is administered the VHTC discussed herein, as well as the dosage which is administered, may be varied to meet one or more user-desired goals or needs.

In various embodiments, the invention comprises a method for attenuating conditions associated with hyperpermeability caused by hemorrhagic shock in mammals comprising raising the threshold for onset of apoptotic processes resultant from the hemorrhagic shock whereby the conditions may be attenuated, wherein raising the threshold for onset of apoptotic processes may comprise preparing in deliverable form a composition comprising an antioxidant, pharmaceutical, regulatory protein, intrinsic mitochondrial regulatory protein, endothelial growth factor, or combinations thereof, and administering an effective amount of said composition to a mammal so as to raise the threshold of apoptosis, wherein the composition may comprise alpha-lipoic acid, Cyclosporin-A, angiopoietin 1, a member of the Bcl-2 family of proteins, non-Bcl-2 proteins, or combinations thereof, wherein conditions may comprise trauma, organ trauma, infections, inflammation, degenerative disease, edema and other conditions associated with hemorrhagic shock, wherein preparing in deliverable form may comprise mixing with a transfection vector, binding to another compound, attaching to an antibody, or combinations thereof, wherein said effective amount of said antioxidant may be about 100 micromole/liter, wherein said mammals may be human beings, wherein said effective amount of a pharmaceutical may be an effective amount to inhibit apoptosis, wherein said effective amount of said endothelial growth factor may be about 200 nanograms/milliliter, wherein said intrinsic mitochondrial regulatory protein from the Bcl-2 family of proteins may comprise Bcl-2, Bcl-xl, MCl-1, A1, Bcl-w, or combinations thereof, and wherein said effective amount of said intrinsic mitochondrial protein to raise the threshold for apoptosis caused by hemorrhagic shock may be at least 2.5 micrograms/milliliter.

In various embodiments, the invention comprises a method for prevention of endothelial cell injury associated with hemorrhagic shock in mammals comprising steps of preparation of an antioxidant in deliverable form, administering an effective amount of said antioxidant to said mammals so as to raise the threshold for apoptosis in endothelial cells, whereby said endothelial cell injury associated with hemorrhagic shock may be prevented, wherein said antioxidant may be alpha lipoic acid, wherein said effective amount of alpha-lipoic acid may be about 100 micromole/liter, wherein said deliverable form may be said antioxidant in a mixture with a transfection vector, and wherein said mammals may be human beings.

In various embodiments, the invention comprises a method for prevention of endothelial cell injury associated with hemorrhagic shock in mammals comprising steps of preparing a pharmaceutical product in a deliverable form, administering said pharmaceutical product in an effective amount to raise the threshold of apoptosis in endothelial cells, whereby endothelial cell injury may be prevented, wherein said pharmaceutical product may be Cyclosporin-A, wherein said effective amount of Cyclosporin-A may be in a range of approximately 5 microliters to 20 microliters per milliliter of blood volume, wherein said deliverable form may be said pharmaceutical product in a mixture with a transfection vector, and wherein said mammal may be a human being.

In various embodiments, the invention comprises a method for prevention of endothelial cell injury associated with hemorrhagic shock in mammals comprising steps of preparing an endothelial growth factor in a deliverable form, administering said endothelial growth factor in an effective amount to increase the threshold for apoptosis in endothelial cells, whereby endothelial cell injury associated with hemorrhagic shock may be prevented, wherein said endothelial growth factor may be angiopoietin-1, wherein said effective amount of angiopoietin-1 may be about 200 nanograms/milliliter, wherein said deliverable form may be said endothelial growth factor in a mixture with a transfection vector, and wherein said mammal may be a human being.

In various embodiments, the invention comprises a method for prevention of endothelial cell injury associated with hemorrhagic shock in mammals comprising steps of preparing an intrinsic mitochondrial regulatory protein in a deliverable form, administering said intrinsic mitochondrial regulatory protein in an effective amount to raise the threshold for apoptosis in endothelial cells, whereby the endothelial cell injury caused by hemorrhagic shock may be prevented, wherein said intrinsic mitochondrial regulatory protein may be Bcl-2, wherein said intrinsic mitochondrial regulatory protein may be Bcl-xl, wherein said intrinsic mitochondrial regulatory protein may be MCl-1, wherein said intrinsic mitochondrial regulatory protein may be A1, wherein said intrinsic mitochondrial regulatory protein may be Bcl-w, wherein said deliverable form may be said intrinsic mitochondrial regulatory protein in a mixture with a transfection vector, wherein said effective amount of intrinsic mitochondrial regulatory protein to raise the threshold for apoptosis caused by hemorrhagic shock may be at least 2.5 micrograms/milliliter, and wherein said mammal may be a human being.

In various embodiments, the invention comprises a method for inhibiting the release of cytochrome c from the mitochondria of endothelial cells during hemorrhagic shock comprising steps of preparing an intrinsic mitochondrial regulatory protein in a deliverable form, administering said intrinsic mitochondrial regulatory protein in an effective amount to raise the threshold for apoptosis in a mammal with hemorrhagic shock, whereby the release of cytochrome c from endothelial cells during hemorrhagic shock may be diminished, wherein said intrinsic mitochondrial regulatory protein may be Bcl-2, wherein said intrinsic mitochondrial regulatory protein may be Bcl-xl, wherein said intrinsic mitochondrial regulatory protein may be MCl-1, wherein said intrinsic mitochondrial regulatory protein may be A1, wherein said intrinsic mitochondrial regulatory protein may be Bcl-w, wherein said deliverable form may be said intrinsic mitochondrial regulatory protein in a mixture with a transfection vector, wherein said effective amount of intrinsic mitochondrial regulatory protein to raise the threshold for apoptosis caused by hemorrhagic shock may be at least 2.5 micrograms/milliliter, and wherein said mammals may be human beings.

In various embodiments, the invention comprises a method for inhibiting induction of caspase-3 from the mitochondria of endothelial cells following hemorrhagic shock in mammals comprising steps of preparing a pharmaceutical product which inhibits induction of caspase-3 in a deliverable form, administering said pharmaceutical product in an effective amount to inhibit the induction of caspase-3 from the mitochondria of endothelial cells following hemorrhagic shock, whereby preventing apoptosis in endothelial cells caused by hemorrhagic shock, wherein said pharmaceutical product may be Cyclosporin-A, wherein said effective amount of Cyclosporin-A may be in a range of approximately 5 microliters to 20 microliters per milliliter, wherein said deliverable form may be said pharmaceutical agent which may inhibit induction of caspase-3 in a mixture with a transfection vector, and wherein said mammals may be human beings.

In various embodiments, the invention comprises a method for inhibiting apoptotic signaling in mitochondria of endothelial cells associated with hemorrhagic shock in mammals comprising steps of preparing an endothelial growth factor in a deliverable form, administering said endothelial growth factor in an effective amount to inhibit apoptotic signaling in mitochondria of endothelial cells, whereby endothelial cell injury associated with hemorrhagic shock may be prevented, wherein said endothelial growth factor may be angiopoietin-1, wherein said effective amount of said angiopoietin-1 may be about 200 nanograms/milliliter, wherein said deliverable form may be said endothelial growth factor in a mixture with a transfection vector, and wherein said mammals may be human beings.

In various embodiments, the invention comprises a method for inhibiting the development of reactive oxygen species by mitochondria of endothelial cells following hemorrhagic shock in mammals comprising steps of preparation of an antioxidant in deliverable form, administering an effective amount of said antioxidant to said mammal so as to prevent the development of reactive oxygen species by the mitochondria of endothelial cells, whereby endothelial cell injury may be prevented, wherein said antioxidant may be alpha-lipoic acid, wherein said effective amount of said alpha lipoic acid may be about 100 micromole/liter, wherein said deliverable form may be said antioxidant in a mixture with a transfection vector, and wherein said mammals may be human beings.

In various embodiments, the invention comprises a method for attenuation of BAK peptide-induced collapse of mitochondrial transmembrane potential caused by hemorrhagic shock in mammals comprising steps of preparing an endothelial growth factor in a deliverable form, administering said endothelial growth factor in an effective amount to attenuate the BAK peptide-induced collapse of mitochondrial transmembrane potential in mammals with hemorrhagic shock, whereby vascular hyperpermeability caused by hemorrhagic shock may be diminished, wherein said endothelial growth factor may be angiopoietin-1, wherein said effective amount of said angiopoietin-1 may be about 200 nanograms/milliliter, wherein said deliverable form may be said endothelial growth factor in a mixture with a transfection vector, and wherein said mammals may be human beings.

In various embodiments, the invention comprises a method for attenuation of the second mitochondrial derived activator of caspases release (smac) caused by hemorrhagic shock in mammals comprising steps of preparing an endothelial growth factor in a deliverable form, administering said endothelial growth factor in an effective amount to attenuate the second mitochondrial derived activator of caspases release (smac) in mammals with hemorrhagic shock, whereby said second mitochondrial derived activator of caspases release (smac) caused by hemorrhagic shock may be diminished, wherein said endothelial growth factor may be angiopoietin-1, wherein said effective amount of said angiopoietin-1 may be about 200 nanograms/milliliter, wherein said deliverable form may be said endothelial growth factor in a mixture with a transfection vector, and wherein said mammals may be human beings.

In various embodiments, the invention comprises a method for inhibition of cytochrome c release caused by hemorrhagic shock in mammals comprising steps of preparing an endothelial growth factor in a deliverable form, administering said endothelial growth factor in an effective amount to inhibit the release of cytochrome c in mammals with hemorrhagic shock, whereby vascular hyperpermeability caused by hemorrhagic shock may be diminished, wherein said endothelial growth factor may be angiopoietin-1, wherein said effective amount of said angiopoietin-1 may be about 200 nanograms/milliliter, wherein said deliverable form may be said endothelial growth factor in a mixture with a transfection vector, and wherein said mammals may be human beings.

In various embodiments, the invention comprises a method for inhibition of caspase-3 activation caused by hemorrhagic shock in mammals comprising steps of preparing an endothelial growth factor in a deliverable form, administering said endothelial growth factor in an effective amount to inhibit the activation of caspase-3 in mammals with hemorrhagic shock, whereby vascular hyperpermeability caused by hemorrhagic shock may be diminished, wherein said endothelial growth factor may be angiopoietin-1, wherein said effective amount of said angiopoietin-1 may be about 200 nanograms/milliliter, wherein said deliverable form may be said endothelial growth factor in a mixture with a transfection vector, and wherein said mammals may be human beings.

In various embodiments, the invention comprises a method for attenuation of vascular hyperpermeability caused by hemorrhagic shock comprising steps of preparing any combination of one or more endothelial growth factors, pharmaceutical agent, antioxidant, and/or intrinsic mitochondrial regulatory protein in a deliverable form, administering said combination of endothelial growth factors, pharmaceutical agent, antioxidant and/or intrinsic mitochondrial regulatory protein in an effective amount, whereby said vascular hyperpermeability may be diminished, wherein said endothelial growth factor may be angiopoietin-1, wherein said pharmaceutical agent may be Cyclosporin-A, wherein said antioxidant may be alpha-lipoic acid, wherein said intrinsic mitochondrial regulatory protein may be Bcl-2, wherein said intrinsic mitochondrial regulatory protein may be Bcl-xl, wherein said intrinsic mitochondrial regulatory protein may be MCl-1, wherein said intrinsic mitochondrial regulatory protein may be A1, and wherein said intrinsic mitochondrial regulatory protein may be Bcl-w.

In various embodiments, the invention comprises a method for prevention of endothelial cell injury caused by hemorrhagic shock comprising steps of preparing any combination of one or more endothelial growth factors, pharmaceutical agent, antioxidant, and/or intrinsic mitochondrial regulatory protein in a deliverable form, administering said combination of endothelial growth factor, pharmaceutical agent, antioxidant and/or intrinsic mitochondrial regulatory protein in an effective amount, whereby endothelial cell injury due to hemorrhagic shock may be prevented, wherein said endothelial growth factor may be angiopoietin-1, wherein said pharmaceutical agent may be Cyclosporin-A, wherein said antioxidant may be alpha-lipoic acid, wherein said intrinsic mitochondrial regulatory proteins may be Bcl-2, wherein said intrinsic mitochondrial regulatory protein may be Bcl-xl, wherein said intrinsic mitochondrial regulatory protein may be MCl-1, wherein said intrinsic mitochondrial regulatory protein may be A1, and wherein said intrinsic mitochondrial regulatory protein may be Bcl-w.

In various embodiments, the invention comprises a method for prolonging an effective therapeutic time period of intrinsic mitochondrial regulatory proteins to raise the threshold of apoptosis in mammals with hemorrhagic shock comprising steps of binding said intrinsic mitochondrial regulatory proteins to another compound, administering said intrinsic mitochondrial regulatory protein bound to another compound to said mammal in hemorrhagic shock, whereby said intrinsic mitochondrial regulatory proteins bound to another compound may attenuate apoptosis of endothelial cells for a longer period of time resulting in diminished vascular hyperpermeability, wherein said intrinsic mitochondrial regulatory protein may comprise Bcl-2, Bcl-xl, MCl-1, A1, Bcl-w, or combinations thereof, wherein said compound to be bound to said intrinsic mitochondrial regulatory proteins may comprise sugars, carbohydrates, nucleotides and polyethylene glycol, or combinations thereof, and wherein said mammals may be human beings.

In various embodiments, the invention comprises a method for delivery of intrinsic mitochondrial regulatory proteins to specific receptors on cell membranes of endothelial cells of mammals with hemorrhagic shock comprising steps of preparing an antibody specific to said specific receptor on the cell membrane of endothelial cells, attaching said intrinsic mitochondrial regulatory protein to said antibody, administering said intrinsic mitochondrial regulatory protein attached to said antibody to said mammal with hemorrhagic shock, whereby said antibody may deliver said intrinsic mitochondrial regulatory protein to said specific receptor causing attenuation of apoptosis and diminished vascular hyperpermeability, wherein said intrinsic mitochondrial regulatory protein may comprise Bcl-2, Bcl-xl, MCl-1, A1, and Bcl-w, or combinations thereof, and wherein said mammals may be human beings.

### Examples

In one or more of the embodiments disclosed herein, the effectiveness of compositions for treating vascular hyperpermeability and methods of administering such compositions is demonstrated. The following embodiments are providing as a demonstration of the function and/or effectiveness of a one or more VHTCs suitably disclosed herein.

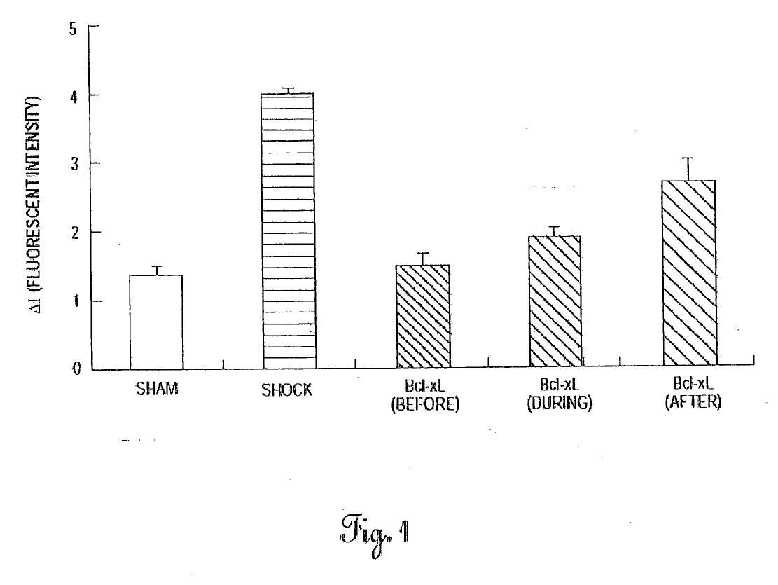

In an embodiment, a member of the Bcl family of proteins may prevent or attenuate endothelial cell dysfunction. For example, in an embodiment of the invention disclosed herein, the Bcl-2 family of proteins, and Bcl-xl in particular, is used to prevent or attenuate endothelial cell dysfunction. This attenuation of apoptosis in endothelial cells maintains the fluid barrier provided by the endothelial cells and prevents or moderates the development of edema through vascular hyperpermeability. For example, in an embodiment of the invention disclosed herein, Sprague-Dawley rats were anesthetized with urethane. Hemorrhagic shock was induced in the anesthetized rats by withdrawing blood to reduce the mean arterial pressure to 40 mm Hg for one hour. The rats were then resuscitated to 90 mmHg by administration of the shed blood and normal saline. Albumin labeled with fluorescein isothiocyanate (FITC) was given intravenously during the period in which shock was present. The mesenteric postcapillary venules in a transilluminated segment of small intestine were examined to quantitate changes in albumin flux using intravital microscopy. Recombinant Bcl-xl was suspended in a standard transfection vector and was given intravenously in an amount of approximately 2.5 microgram/ml of the total rat blood volume, before, during or after hemorrhagic shock in three separate groups of rats to determine endothelial cell integrity. Cytosolic cytochrome c levels and caspase-3 activity were also determined in mesenteric tissue collected from the animals after Bcl-xl transfection and hemorrhagic shock. As shown in FIG. 1, the administration of the protein Bcl-xl to the traumatized rats attenuated the degree of hemorrhagic shock-induced hyperpermeability. The degree of attenuation in hyperpermeability afforded by administration of Bcl-xl was greatest when Bcl-xl was administered prior to the onset of shock. Treatment of rats with Bcl-xl during the course of induced hemorrhagic shock resulted in a greater decrease in vascular hyperpermeability than did treatment with Bcl-xl after the shock period was over. A mechanism of action of the Bcl-2 family of proteins in general, and Bcl-xl, in particular, is to prevent release of cytochrome c from the mitochondrion following the onset of hemorrhagic shock. Preventing the release of cytochrome c from the mitochondria breaks the pathway to apoptosis resulting in prevention of injury to endothelial cells. Prevention of injury to endothelial cells results in an attenuation of vascular hyperpermeability during periods of hemorrhagic shock.

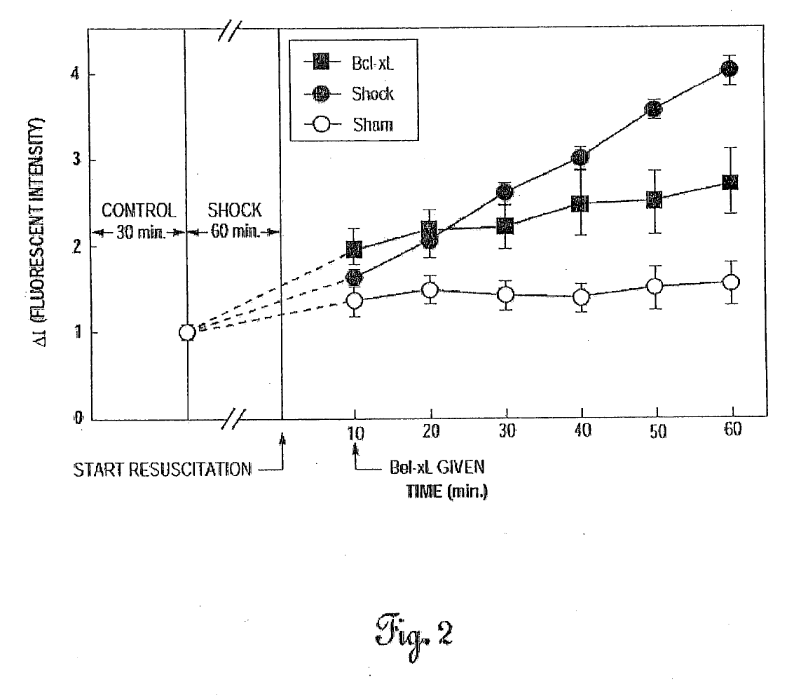

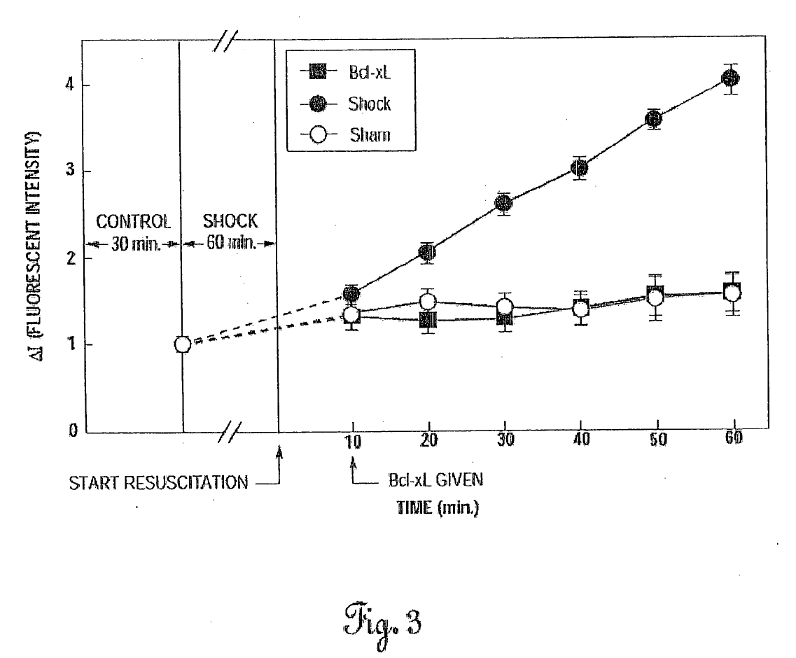

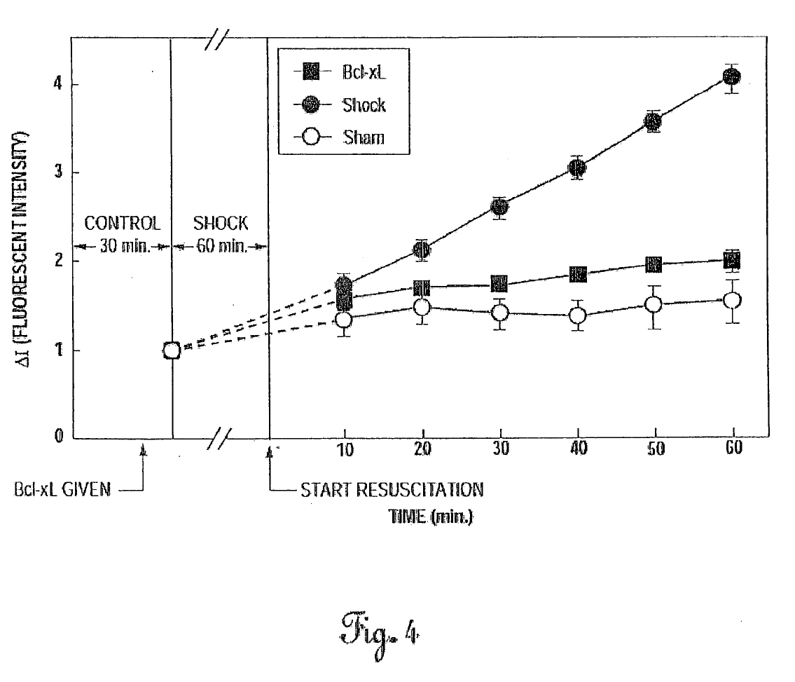

In another embodiment, a member of the Bcl family of proteins may attenuate vascular hyperpermeability. For example, in an embodiment a Bcl-xl given after one hour of shock and 10 minutes of resuscitation attenuated vascular hyperpermeability as compared to untreated animals as shown in FIG. 2. This finding confirms that intravenous administration of the intrinsic mitochondrial regulatory protein, Bcl-xl, after the onset of shock, can diminish the amount of vascular hyperpermeability. In another embodiment of the invention disclosed herein and demonstrated in FIG. 3, administration of Bcl-xl during the period of shock, but before resuscitation efforts are started, almost eliminated the hemorrhagic shock-induced hyperpermeability. In addition, Bcl-xl was given after the shock period during resuscitation and effectively reversed the hyperpermeability induced by hemorrhagic shock. These findings support the use of the intrinsic mitochondrial regulatory protein, Bcl-xl, as a “front-line” treatment of hemorrhagic shock. In yet another embodiment, hemorrhagic shock-induced hyperpermeability was almost eliminated when rats were treated with Bcl-xl prior to the onset of shock as shown in FIG. 4.

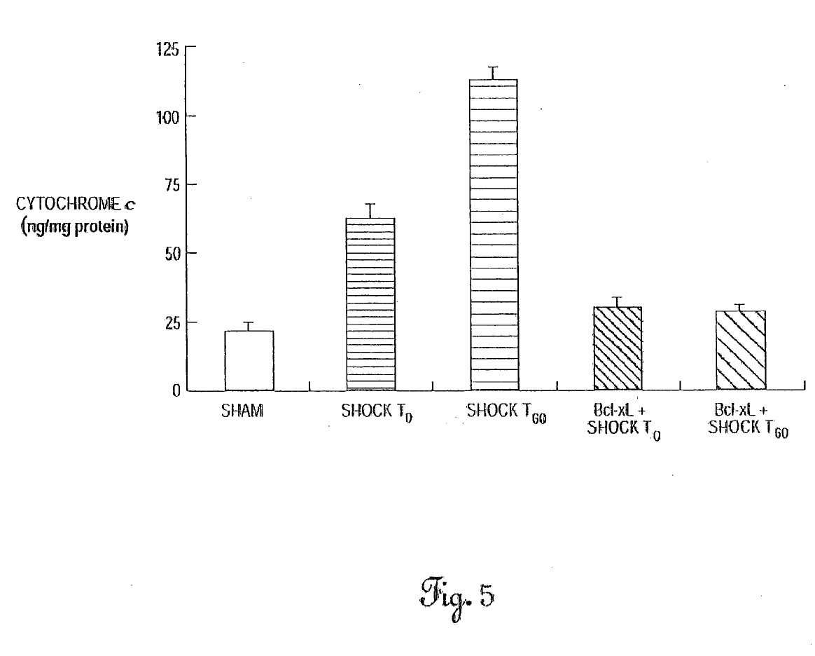

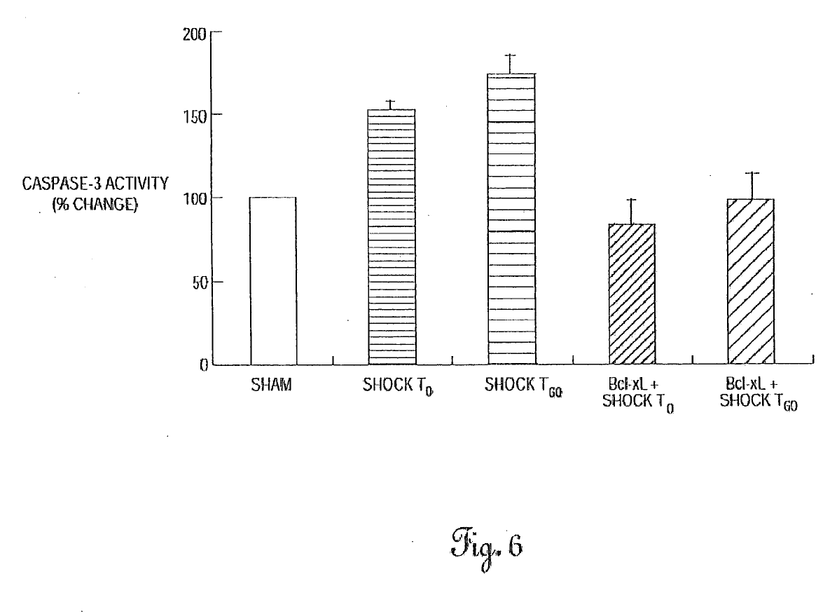

In another embodiment, a member of the Bcl family of proteins may inhibit the release of cytochrome c. For example, in another embodiment the administration of Bcl-xl inhibited the release of cytochrome c into the cytoplasm from the mitochondria following hemorrhagic shock as shown in FIG. 5. FIG. 6 demonstrates another embodiment of the invention disclosed herein. Administration of Bcl-xl reduced the activation of caspase-3 following hemorrhagic shock. As described above both cytochrome c and caspase-3 play vital roles in the regulation and initiation of apoptosis of endothelial cells following hemorrhagic shock.

In one or more of the aforementioned embodiments, Bcl-xl was disclosed as having the property of inhibiting apoptosis as measured by attenuation of vascular hyperpermeability, a decrease in cytochrome c release and reduction in caspase-3 activity following administration of Bcl-xl. The use of Bcl-xl to prevent or diminish the degree of edema following trauma in mammals is clearly indicated. The other members of the Bcl-2 family of proteins, such as BAX, BAK, MCL-1, A1 and BCL-W may also have useful properties of preventing edema as does Bcl-xl and are specifically disclosed as such, herein.

In an embodiment, the protein Bcl-xl may be administered to the test animals in the aforementioned embodiments by transfection. Standard transfection vectors, such as “transIT” and “chariot,” may be useful in facilitating entry of the intrinsic mitochondrial regulatory proteins and other substances which are disclosed herein through the membrane of the endothelial cell into the cytoplasm of the endothelial cell where regulation of apoptosis at the level of the mitochondrion can take place. The use of transfection to deliver Bcl-xl to the test animals was not meant to exclude other methods of delivery that are well known to those of ordinary skill in the art. For example, the intrinsic mitochondrial regulatory proteins could be bound to antibody or antigen-recognizing fragments of antibody which are specifically directed to receptor proteins on the cell membrane of endothelial cells. In this manner, the intrinsic mitochondrial regulatory protein could be delivered directly to the endothelial cell. Nonlimiting examples of other delivery methods include plasmid vectors, viral vectors, liposomes, antibody vectors, and others which are included in this disclosure as if specifically set forth. In an alternative embodiment, a Bcl-family protein may be administered absent a delivery vehicle.

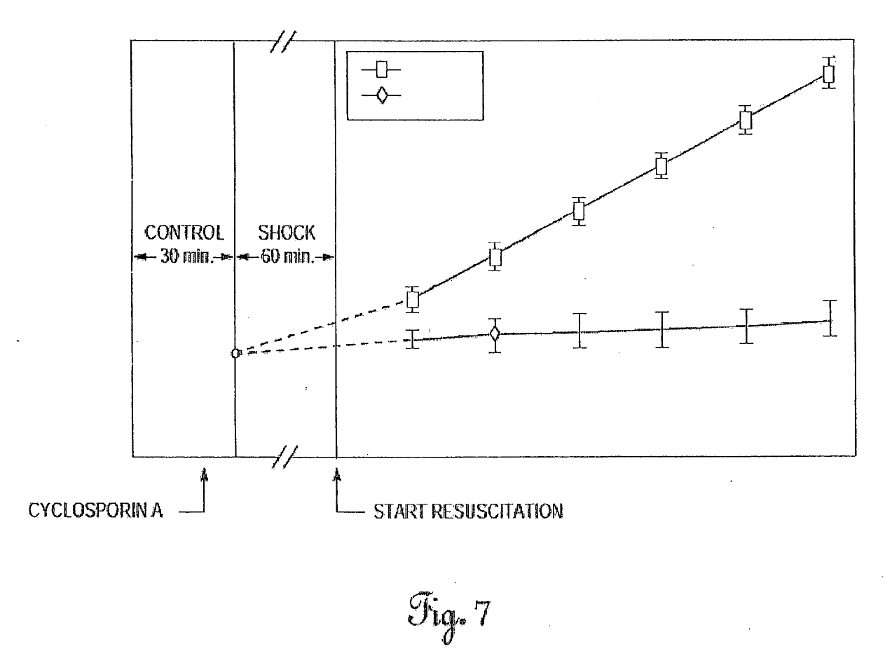

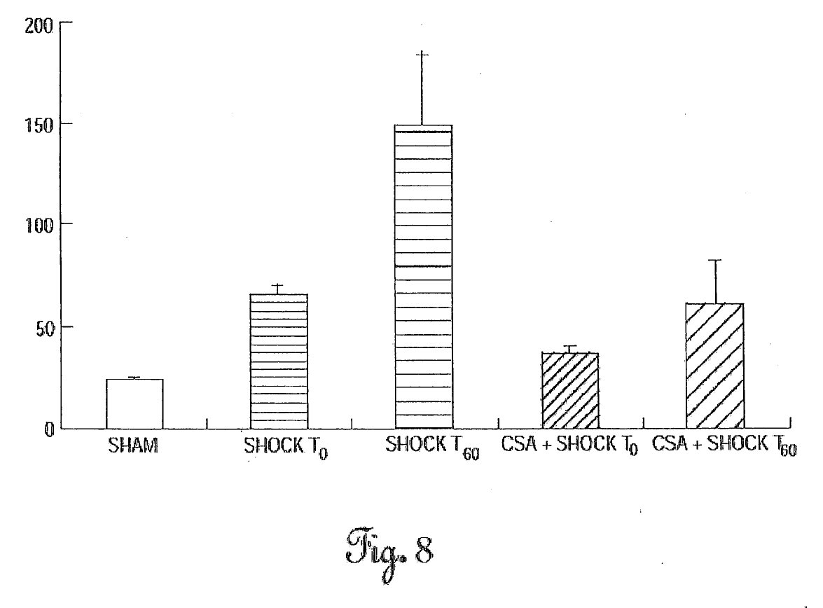

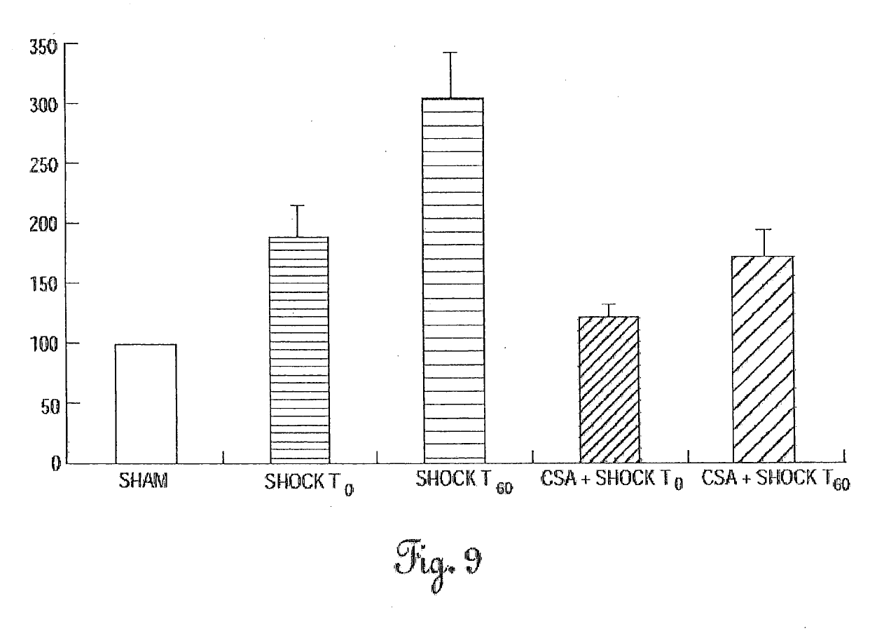

In an embodiment, other apoptotic modulators may include mediators of the immune response such as Cyclosporin-A used initially to prevent rejection of transplanted organs, also affect apoptosis of endothelial cells as shown in FIGS. 7, 8 and 9. For example, in this embodiment of the invention disclosed herein, the administration of Cyclosporin-A by transfection, for example, prior to the induction of shock in rats as described above, resulted in a complete elimination of vascular hyperpermeability as shown in FIG. 7. That Cyclosporin-A exerts its effect on vascular hyperpermeability by inhibiting apoptosis of endothelial cells is shown in FIG. 8 and FIG. 9 wherein administration of Cyclosporin-A inhibits cytochrome c release from mitochondria and diminishes the induction of caspase-3 activity by hemorrhagic shock, respectively. Cyclosporin-A is effective in preventing edema in mammals following acute trauma. The amount of Cyclosporin-A administered to traumatized animals is an amount which effectively inhibits apoptosis and is in a range of approximately 5 microliters to approximately 20 microliters per milliliter of blood volume.

Because of the role of ROS in the development of cell permeability following hemorrhagic shock, antioxidants were employed to inhibit the development of ROS and minimize the development of cell permeability and cell injury related to the development of ROS during apoptosis. In this embodiment of the invention disclosed herein, antioxidants such as alpha-lipoic acid were administered to animals traumatized as described above. The administration of alphalipoic acid attenuated the amount of vascular hyperpermeability induced by hemorrhagic shock-induced apoptosis. Alpha-lipoic acid administered by transfection in a dosage of about 100 mg/kg was effective in reducing the amount of vascular hyperpermeability if administered either before the onset of hemorrhagic shock or within 60 minutes after the development of hemorrhagic shock.

In another embodiment of the invention described herein, it is disclosed that angiopoietin-1, an endothelial cell growth factor, administered to mammals with hemorrhagic shock, attenuated the amount of vascular hyperpermeability demonstrated by those traumatized animals. Angiopoietin-1 administered intravenously in a dosage of 200 ng/ml to traumatized animals attenuated the amount of vascular hyperpermeability observed in those animals. The effect of angiopoietin-1 on lessening vascular hyperpermeability was to disrupt the apoptotic signaling mechanism which initiates and sustains the process of apoptosis by inhibiting one or a combination of factors comprising: (1) BAK peptide-induced collapse of mitochondrial transmembrane potential, (2) second mitochondrial derived activator of caspases release (smac), (3) cytochrome c release, and (4) activation of caspase-3.

As described above, intrinsic mitochondrial regulatory proteins were administered intravenously to traumatized animals. It is further disclosed herein that the intrinsic mitochondrial regulatory proteins may be administered by other routes, including, but not limited to, the sublingual route, direct injection into a body cavity or through the peritoneum into the abdominal cavity. Administration of the intrinsic mitochondrial regulatory proteins by these other avenues would raise the threshold of apoptosis and prevent vascular hyperpermeability and edema.

When foreign proteins are injected into a mammal, the host animal recognizes the proteins as foreign and attempts to eliminate them quickly from the body of the host. This rapid elimination of these administered proteins can diminish the activity of those administered proteins and deprive the host animal with their full benefit. This removal of administered proteins can be inhibited to some extent by binding to the foreign proteins substances which slow or prevent the process of natural elimination of foreign proteins. It is specifically disclosed herein, that the intrinsic mitochondrial regulatory proteins can be specifically attached to other compounds prior to administration to the traumatized animal which prolongs the effective time period in which the intrinsic mitochondrial regulatory protein can act to inhibit apoptosis in endothelial cells of traumatized animals. Those substances which can be attached to the intrinsic mitochondrial regulatory proteins to prolong their presence in the animal's circulation include but are not limited to sugars, carbohydrates, nucleotides, polyethylene glycol and the like.

The invention disclosed herein is a method for treatment of patients with edema following the development of shock. The method comprises modulating the apoptotic process in the endothelial cells lining the lumen of small venules, capillaries and other vascular structures, in order to preserve the barrier to leakage of fluid from the blood to the other tissues and prevent or diminish edema. This amelioration of edema would prevent organ failure and promote the effectiveness of resuscitation measures used to treat shock. As shown above, regulatory proteins, pharmaceuticals, antioxidants, endothelial growth factors, and other compounds and processes related to regulation of apoptosis can be modulated to prevent the death of endothelial cells and development of edema. In particular and in various embodiments, manipulation of the Bcl-2 family of proteins, immunomodulating compounds such as Cyclosporin-A, endothelial growth factors such as angiopoietin-1, and antioxidants such as deprenyl or alpha-lipoic acid, provide such desirable results. Administration of such compounds to trauma patients, either alone or in combination, would save many lives and prevent other co-morbidities caused by the organ damage associated with edema resulting from vascular hyperpermeability. Administration of a combination of the apoptotic modulators described above would inhibit the apoptotic cascade at different points making the use of a combination of the aforementioned apoptotic modulators an effective inhibitor of vascular permeability caused by endothelial cell death. In an embodiment, a combination of apoptotic modulators suitable for use in this disclosure comprises an intrinsic regulatory protein, an immune modulator and an antioxidant. In an alternative embodiment, a combination of apoptotic modulators suitable for use in this disclosure comprises antiapoptotic protein, such as Bcl-2, Bcl-xl, MCl-1, A1 and Bcl-w, or an anti-proapoptotic protein, such as an inhibitor or antibody to a proapoptotic protein, such as BAK and BAX-1 combined with an immune or mitochondrial modulator, such as Cyclosporin-A, estradiol, or angiopoietin 1, and/or an antioxidant, such as deprenyl or alpha-lipoic acid.

What is claimed is:

1. A method comprising administering in a form deliverable to a mammal a composition comprising an apoptosis regulating protein, antioxidant, mitochondrial modulator, biological effector molecule or combinations thereof in an amount effective to raise the threshold for apoptosis, whereby raising the threshold for apoptosis prevents vascular hyperpermeability.

2. The method of claim 1 wherein the composition is anti-apoptotic.

3. The method of claim 1 wherein the protein comprises an isolated and/or purified protein from the Bcl family of proteins, or functional derivatives thereof.

4. The method of claim 1 wherein the composition comprises SEQ ID NO:1, SEQ ID NO:2, or combinations thereof.

5. The method of claim 1 wherein the composition comprises a fragment of SEQ ID NO:1, SEQ ID NO:2, or combinations thereof.

6. The method of claim 1 wherein the antioxidant comprises ascorbic acid, glutathione, uric acid, carotenoids, α-tocopherol, ubiquinol, diprenyl, or combinations thereof.

7. The method of claim 1 wherein the mitochondrial modulator comprises an immunomodulatory agent.

8. The method of claim 1 wherein the mitochondrial modulator comprises Cyclosporin A, Tacrolimus or combinations thereof.

9. The method of claim 1 wherein the composition further comprises a pharmaceutically acceptable carrier or excipient.

10. The method of claim 1 wherein the components of the composition are administered individually, as a single unitary preparation, or combinations thereof.

11. The method of claim 1 further comprising co-administering the composition and a conventional treatment for hemorrhagic shock to a mammal in need thereof.

12. The method of claim 11 wherein conventional treatment comprises the administration of plasma.

13. The method of claim 12 wherein an amount of plasma administered is reduced compared to an otherwise similar method lacking the composition.

14. The method of claim 1 wherein the biological effector molecule comprises an endothelial growth factor.

15. The method of claim 14 wherein the endothelial growth factor elicits gene activation, cell proliferation, cell differentiation, matrix dissolution stimulation of regulatory cascades leading to angiogenesis, cellular migration, and/or degradation of matrix metalloproteinase (MMP), or combinations thereof.

16. The method of claim 14 wherein the endothelial growth factor stimulates angiogenesis.

17. A pharmaceutical composition comprising: an antioxidant, a mitochondrial modulator, an apoptosis regulating protein, a biological effector molecule, or combinations thereof, wherein the composition is effective for diminishing hyperpermeability by modulating apoptotic signaling in mammalian endothelial cells.

18. The pharmaceutical composition of claim 17 further comprising a transfection agent, wherein the transfection agent is effective to achieve delivery of at least a portion of the composition to the endothelial cells.

19. The composition of claim 17 comprising α-lipoic acid, angiopoietin 1, Cyclosporin-A, a protein from the Bcl-family of proteins, or combinations thereof.

20. An article of manufacture comprising the pharmaceutical composition of claim 17.
